(19) United States
(12) Patent Application Publication
(10) Pub. No.: US 2016/0194272 A1
(43) Pub. Date: Jul. 7, 2016

(54) PROCESS FOR THE ETHERIFICATION OF AMINO ALCOHOLS

(71) Applicant: BASF SE, Ludwigshafen am Rhein (DE)

(72) Inventors: Melanie WEINGARTEN, Ratzeburg (DE); Wolfgang SIEGEL, Limburgerhof (DE); Michael SIEMER, Mannheim (DE); Junmin JI, Beaumont, TX (US); Joseph VALCHO, Naperville, IL (US)

(73) Assignee: BASF SE, Ludwigshafen am Rhein (DE)

(21) Appl. No.: 14/963,948

(22) Filed: Dec. 9, 2015

Related U.S. Application Data
(60) Provisional application No. 62/090,898, filed on Dec. 12, 2014.

(51) Int. Cl. C07C 213/06

(52) U.S. Cl. CPC C07C 213/06; C07B 2200/07

(57) ABSTRACT

A process for the preparation of the ether of formula I

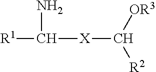

where R₁ and R₂ independently from one another are hydrogen or an alkyl group with 1 to 10 C atoms, R₃ is an alkyl group with 1 to 10 carbon atoms and
X is a bond or a hydrocarbon group with 1 to 10 carbon atoms
comprising
a) deprotonating the amino alcohol of formula II

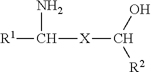

where R₁, R₂ and X have the meaning above
with a metal as deprotonating agent to give the anion of formula III

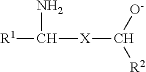

where R₁, R₂ and X have the meaning above
and
b) alkylation of the anion obtained in step a) with an alkylation agent to give the ether of formula I.

The present invention relates a process for the preparation of the ether of formula I

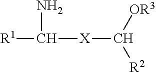

where R1 and R2 independently from one another are hydrogen or an alkyl group with 1 to 10 C atoms, R3 is an alkyl group with 1 to 10 carbon atoms and
X is a bond or a hydrocarbon group with 1 to 10 carbon atoms
comprising
a) deprotonating the amino alcohol of formula II

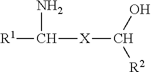

where R1, R2 and X have the meaning above
with a metal as deprotonating agent to give the anion of formula III

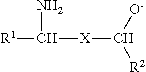

where R1, R2 and X have the meaning above
and
b) alkylation of the anion obtained in step a) with an alkylation agent to give the ether of formula I.

Ethers of formula I are chemical intermediates which are, for example, used for the synthesis of pharmaceuticals, plant protecting agents as herbicides, insecticides or fungicides.

Compounds of formula I may be obtained by etherification of amino alcohols. As such amino alcohols have two functional groups (a primary amino group and a hydroxy group) a selective etherification of the hydroxy group becomes problematic. Mono- or di-alkylation of the nitrogen atom will occur sometimes even preferentially. In particular the by-product with a mono-alkylated nitrogen has to be avoided as the boiling point of such by-product is quite similar to the boiling point of the desired ether of formula I. Hence any separation of such by-product by distillation becomes difficult.

In addition it is often required that the compound of formula I is a defined stereo isomer. Hence an amino alcohol with a defined stereo isomerism is selected as starting material, for example a pure (S) or (R) isomer. Any isomerization during the preparation of the ether has to be avoided and the ether obtained should finally have the same stereo isomerism as the amino alcohol.

DE-A 103 44 447 discloses a process for the etherification of amino alcohols having an unsubstituted amino group and a hydroxyl group. The process described is a two-step process. In a first step an alkali alcoholate is used to prepare the alcoholate of the amino alcohol. In a second step the alcoholate of the amino alcohol is alkylated with an alkylating agent to finally form the corresponding ether.

It is the object of the present invention to improve the process for the preparation of ethers of formula I. The improved process should be very effective and easy to perform. The yield of amino alcohol ethers should be high and any by-products, specifically by-products with substitution at the nitrogen should be avoided. The overall selectivity of the ethers and the retention of the stereo chemistry should be as high as possible.

Accordingly, a process as defined above has been found.

To the ether for formula I

This claimed process is a process for the preparation of ether of formula I

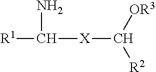

where R₁ and R₂ independently from one another are hydrogen or an alkyl group with 1 to 10 C atoms, R₃ is an alkyl group with 1 to 10 carbon atoms and X is a bond or a hydrocarbon group with 1 to 10 carbon atoms.

Preferably R₁ and R₂ independently from each other are hydrogen or an alkyl group with 1 to 4 C atoms.

Preferably R₃ is an alkyl group with 1 to 4 C atoms.

Preferably X is a bond or an alkylene group with 1 to 10 carbon atoms, in a particularly preferred embodiment X is a bond.

In a particularly preferred embodiment the compound of formula I is a compound wherein R₁ is hydrogen or an alkyl group with 1 to 4 C atoms, R₂ is hydrogen, R₃ is an alkyl group with 1 to 4 C atoms and X is a bond or an alkylene group with 1 to 10 carbon atoms.

In a most preferred embodiment the compound of formula I is a compound wherein R₁ is a methyl group, R₂ is hydrogen, R₃ is a methyl group and X is a bond. This compound is known as 1-methoxy-2-propylamin.

Preferably the compound of formula I has a defined stereo isomerism. In particular it may be a (R) or (S) enantiomer or a defined mixture thereof, the chiral carbon atom being the carbon atom to which the primary amino group is bonded. Particularly preferred is a pure (R) or (S) enantiomer.

In a most preferred embodiment the compound of formula I is (S)-1-methoxy-2-propylamin.

To process step a)

In process step a) an amino alcohol of formula II

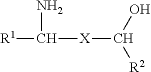

is deprotonated with a metal as deprotonating agent to give the anion of formula III

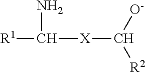

In both formulas II and III R₁, R₂, R₃ and X have the meanings and preferred meanings above.

In the most preferred embodiment the amino alcohol of formula II is alaninol, in particular pure (S)-alaninol (R₁=Methyl, R₂═H, X=bond)

The metal might for example be a metal of any of groups I to III of the periodic system.

Preferably the metal is an alkali or earth alkali metal.

In a particularly preferred embodiment the metal is an alkali metal, for example lithium, sodium or potassium.

In a most preferred embodiment the metal is sodium.

Process step a) may be performed in presence of a solvent or in the absence of a solvent.

In the absence of a solvent the amino alcohol (formula II) and/or the ether obtained (formula I) would have partially the function of a solvent.

In a preferred embodiment step a) is performed in the presence of a solvent.

Suitable solvents are, for example, aliphatic solvents, for example C5- to C16-alkanes like hexane, cyclohexane, heptane, aromatic solvents, including alkyl & alkoxyaromatics, like toluene or anisol, ether including cyclic ethers, e.g. dioxane, tetrahydrofurane, or ethyleneoxide ethers, in particular glymes like 1,2-dimethoxyethane (monoglyme), diethyleneglycoldimethylether (diglyme), 1,2-diethoxyethane (ethylglyme), diethoxy-diethylene glycol (ethyl diglyme), diethylene glycol dibutylether (butyl diglyme) et al.) or polyethers like poly(ethylene glycol) dimethylether et al., or alkylamines (mono-, di-, trialkylamines, C5-C10 like tert-butylamine or tribuylamine).

Preferred solvents are hydrophobic solvents, in particular aliphatic or aromatic hydrocarbons or ethers, as for example C7- to C15 alkanes, alkylaromatic solvents or ethyleneoxide ethers.

More preferred are aromatic solvents, in particular aromatic hydrocarbons like toluene or xylene and ether.

Most preferred solvents are ether, in particular ether of formula IV

Rₐ—O—(CH₂—O—)ₙR_{b}

wherein Rₐ and R_{b} are a C1- to C6-alkyl group and n is an integral number from 1 to 4.

Particularly preferred are ethers of formula IV with n=2, known as diglyme; most preferred is butyldiglyme (Rₐ and R_{b} are butyl).

Preferably a mixture of the amino alcohol and a solvent is used in process step a).

Preferably, the amino alcohol, respectively the mixture, should be free of water and other protic solvents.

The metal may be used in solid or liquid (molten) form. The metal may be added to the amino alcohol, the solvent or the mixture thereof.

In a preferred embodiment a mixture of the amino alcohol in a solvent is prepared first and the liquid (molten) metal added thereafter.

Preferably the deprotonating reaction is performed at a temperature where the metal is liquid. In a particularly preferred embodiment the metal is melted to become liquid and is added as liquid to the aminoalcohol or mixture.

Process step a) is preferably performed at a temperature of at least the melting temperature of the metal used as deprotonating agent.

The temperature may be, for example, at minimum 40° C., preferably at least 60° C., respectively 80° C. The temperature may be, for example, at maximum 200° C., respectively 160° C. or 140° C. A very suitable range of temperatures is, for example 80 to 120° C.

Process step a) may be performed at normal, at reduced or at increased pressure. Usually process step a) will be performed at a pressure of 0.4 to 3 bars, in particular at 0.8 to 1.5 bar and preferably simply at normal pressure (1 bar).

Preferably the metal should be consumed in process step a). Hence, in a preferred embodiment the amino alcohol and the metal are used in equimolar amounts or—even more preferred—the amino alcohol is used in excess. The ratio of equivalents metal to amino alcohol could for example be from 1:1 to 1:2, in particular from 1:1.05 to 1:1.5.

To process step b)

In process step b) the anion of formula III obtained in step a) is alkylated. The reaction under step b) is preferably started when all metal in process step a) is consumed.

Suitable alkylating agents are well known. Usual alkylating agents correspond to the general formula V

(Alkyl)ₘ-Z,

wherein Alkyl is an alkyl group, preferably an alkyl group with 1 to 4 carbon atoms, most preferred a methyl group, m may be 1, 2 or 3 and Z is a one, two or three valent inorganic or organic, corresponding the actual meaning of n.

In particular n is 1 or 2.

In particular Z is a halogen, for example chloride, or an organic or inorganic ester group.

As examples alkyl chloride, alkyl mesylate, alkyl tosylate, dialkyl sulfate or dialkyl carbonate, trialkyl phosphate may be named.

As R₃ preferably is a methyl group, the preferred reaction is a methylating reaction using, for example, methyl chloride, dimethyl sulfate or dimethyl carbonate as methylating agent.

In one preferred embodiment the alkylating agent, respectively methylating agent, is used as a gas. The boiling point of methyl chloride is −23.8° C. at 1 bar.

In a preferred embodiment of step a) a solvent has been used. This solvent is preferably not removed in or before step b). Hence both, process step a) and b) are preferably performed in presence of the same solvent, which is most preferred an ether of formula IV, in particular butyldiglyme.

In reaction step b) the reactants may be used in equimolar amounts or either reactant may be used in excess.

In particular the ratio of equivalents alkylating agent to the equivalents metal used in process step a) may for example be from 0.5:1 to 1:0.5.

Preferably the ratio of equivalents alkylating agent to the equivalents metal is from 0.5:1 to 0.99:1, even more preferred is a ratio from 0.9:1 to 0.99:1.

Process step b) may be performed at elevated temperature.

The temperature may be, for example, at minimum 30° C., respectively 60° C. The temperature may be at maximum 200° C., respectively 160° C. or 140° C., in particular at maximum 120° C. A very suitable range of temperatures is, for example 60 to 120° C., respectively 70 to 100° C.

Process step b) may be performed at normal, at reduced or at increased pressure. Process step b) may be for example performed at a pressure of 0.4 to 3 bars, in particular at 0.8 to 1.5 bar and preferably simply at normal pressure (1 bar).

Process step b) usually results in a suspension comprising the ether of formula I, salts that have been formed from the metal cation and the remaining group of the alkylating agent, for example sodium chloride, organic solvent that has been used and further by-products.

The suspension usually comprises solid salts. In order to withdraw such salts from said suspension, the salts may be filtered off. Such filtration would usually be done before any distillation.

The ether of formula I may be distilled from such suspension or solution and thereafter purified, for example by further distillation.

Alternatively the salts may be removed after the distillation of the ether by adding an amount of water which is sufficient to solve any salts. The obtained aqueous salt solution and the solvent form distinct phases and can be separated easily.

By-products that may have been formed are compound with substitution at the nitrogen atoms, in particular compounds wherein the nitrogen atom is alkylated as well, either once (giving a secondary amino group) or even twice (giving a tertiary amino group).

It is advantage of the claimed process that such by-products are not or at least hardly formed.

In particular the molar ratio of the ether of formula I to the by-product which is N-mono alkylated as well is usually more than 15:1, preferably more than 20:1.

The selectivity of the ether of formula I is usually more than 90%, in particular more than 95%, respectively more than 97%, such selectivity being the ratio of the equivalents of the ether of formula I to the sum of the equivalents of all and any alkylated compounds×100%.

It is a further advantage of the claimed process that the stereo isomerism of the amino alcohol is kept. Usually at least 90%, typically at least 95%, respectively at least 98% of the ether of formula I obtained by the process has the same stereoisomerism as the amino alcohol. Hence starting with (S)-alaninol will give by methylating at least 90%, typically at least 95%, respectively at least 98% (S)-1-methoxy-2-propylamin.

Furthermore the process claimed is a very effective and efficient process. The process allows high yields of ether. According to the state of the art metal alcoholates like sodium methylate are used as deprotonating agent. With sodium methylate methanol is set free. The process claimed is simplified compared to the prior art as an additional solvent resulting from the use of metal alcoholates, for example methanol, is avoided.

### EXAMPLES

MOIPA shall be any 1-methoxy-2-propylamin

(S)-MOIPA shall be the stereo isomer (S)-1-methoxy-2-propylamin.

N-Me-MOIPA shall be (S)-1-methoxy-2-propylamin with an additional methylation at the nitrogen atom ((2S)-1methoxy-N-methyl-propan-2-amine (IUPAC-name).

Gas chromatography (Hewlett Packard 6890 N with FID detector; column: 25 m Hydrodex-γ-TBDAc, inner diameter 0.25 mm, outer diameter 0.40 mm, film diameter 0.25 μm, oven program: temperature start: 60° C., hold: 0 min, in 5 K/min steps to temperature end: 220° C., hold: 1 min; column: 30 m Optima 5 MS, inner diameter 0.25 mm, outer diameter 0.45 mm, film diameter 1.0 μm, oven program: temperature start: 60° C., hold: 5 min, in 15 K/min steps to temperature end: 280° C., hold: 1 min.) was used to determine the yield and selectivity. The yield and selectivity were calculated from the area of the peaks of the gas chromatogram using calibration methods.

### Example 1

2652 g of diethylene glycol dibutylether (12.15 mol) were added to a 4 liter reactor (with Rushton type impeller and pitched-blade impeller) and brought to about 100° C. (temperature (jacket)=110° C.). 192 g of sodium (8.34 mol) were added over 20 min at about 100° C. in small pieces until the sodium was all molten and emulsified in relatively small sphericals. The reaction mixture was stirred for further 30 min at 105° C. and 150 rpm (rounds per minute). A clear yellow-brown solution was obtained with very small sphericals of sodium.

663 g of L-alaninol (8.77 mol) were added drop wise under control of the hydrogen evolution and temperature during 1 h (hour) and 20 min at about 104 to 121° C. A grey-white suspension was obtained. The reaction mixture was stirred for further 6 h at 320 rpm and a temperature of 110° C.

412 g of methylchloride (8.16 mol) gas were inserted during 15 h and 10 min at 98 to 103° C. and 500-900 rpm into the suspension (velocity of gas insertion 23-33 g).

The composition of the product mixture obtained was analyzed.

More than 99.9% of the MOIPA obtained were (S)-MOIPA.

The yield of (S)-MOIPA was 87% (based on methyl chloride).

The ratio of (S)-MOIPA to the byproduct N-Me-MOIPA was 22:1 (ratio of the corresponding areas of the peaks of the gas chromatogram).

The selectivity of(S)-MOIPA was 93.8%

The (S)-MOIPA was removed from the reaction mixture by distillation.

Distillation was performed at 100 mbar, 300 rpm and no distillation column was used for this crude first distillation. In total, 675.3 g of distillate were obtained with a MOIPA content of 93.6 weight % which corresponds to a distillation yield of more than 99%. The residue after distillation contains less than 2 weight % MOIPA. The distillate was further purified by distillation to obtain (S)-MOIPA in a purity of 99. 92 GC-area %: 0.02 GC-N-Me-MOIPA, other organics found were 0.01 GC-area % and 0.04 GC-area %, the water content was determined to be 0.08 weight %.

1603 g of distilled water were added to the residue of the distillation in order to dissolve the sodium chloride and to provide a saturated brine solution. The mixture was stirred at 50° C. for 30 min and after further 6 min the phases were separated. 2000 g of an aqueous phase containing 72.4 weight % water and 2672 g of organic phase containing 1.17 weight % of water were obtained.

### Example 2

2652 g of diethylene glycol dibutylether (12.15 mol) were added to a 4 liter reactor (with Rushton type impeller and pitched-blade impeller) and brought to about 100° C. (temperature (jacket)=110° C.). 192 g of sodium (8.34 mol) were added over 15 min at about 100° C. in small pieces until the sodium was all molten and emulsified in relatively small sphericals. The reaction mixture was stirred for further 20 min at 105° C. and 150 rpm (rounds per minute). A clear yellow-brown solution was obtained with very small sphericals of sodium.

663 g of L-alaninol (8.77 mol) were added drop wise under control of the hydrogen evolution and temperature during 1 h (hour) and 40 min at about 105-116° C. A grey-white suspension was obtained. The reaction mixture was stirred for further 6 h at 400 rpm and a temperature of 110° C.

412 g of methylchloride (8.16 mol) gas were inserted during 21 h at 96-102° C. and 500 rpm into the suspension (velocity of gas insertion 16-21 g/h).

The composition of the product mixture obtained was analyzed.

99.3% of the MOIPA obtained were (S)-MOIPA.

The yield of (S)-MOIPA was 75% (based on methyl chloride).

The ratio of (S)-MOIPA to the byproduct N-Me-MOIPA was 28:1.

The selectivity of (S)-MOIPA was 94.5% (S)-MOIPA.

The (S)-MOIPA was removed from the reaction mixture by distillation.

Distillation was performed at 150 mbar, 300 rpm and no distillation column was used for this crude first distillation. In total, 521.5 g of distillate were obtained with a MOIPA content of 95.3 weight % which corresponds to a distillation yield of 96%. The residue after distillation contains less than 2 weight % MOIPA. The distillate was further purified by distillation to obtain (S)-MOIPA in a purity of 99.92 GC-area %:0.02 GC-N-Me-MOIPA, other organics found were 0.01 GC-area % and 0.04 GC-area %, the water content was determined to be 0.08 weight %.

1320 g of distilled water were added to the residue of the distillation in order to dissolve the sodium chloride and to provide a sat. brine solution. The mixture was stirred at room temperature for 1 h and after additional 7 min the phases were separated. 1729 g of an aqueous phase containing 73.7 weight % water and 2386 g of organic phase containing 0.94 weight % of water were obtained.

### Example 3

187 mL of o-xylene (1.53 mol) were added to a 0.5 L flask and brought to 100-105° C. 11.63 g of sodium (0.51 mol) were added over 30 min in small pieces until the sodium was all molten and emulsified in relatively small sphericals. The reaction mixture was stirred at 850 rpm for further 30 min.

40 g of L-Alaninol (0.53 mol) were added dropwise under control of the hydrogen evolution and temperature during 1 h and 30 min at 98-105° C. and 1000 rpm. A white very thick suspension was obtained. The reaction mixture was stirred for further 1 h at 100° C. A white stirrable suspension was obtained that was brought to 85° C.

25 g of methylchloride gas (0.50 mol) were inserted using a gassing stirrer. Gas insertion was performed with a velocity of 8-10 g methylchloride/h and after 15% of the methylchloride gas was inserted the temperature was kept at 69-75° C.

The composition of the product mixture obtained was analyzed.

99.5% of the MOIPA obtained were (S)-MOIPA.

The yield of (S)-MOIPA was 73% (based on methyl chloride).

The ratio of (S)-MOIPA to the byproduct N-Me-MOIPA was 30:1.

The selectivity of (S)-MOIPA was 92.9%.

Crude overhead distillation (using a 50 cm column packed with a V2A filling, 3×3 mm and a Normag) of (S)-MOIPA, N-Me-MOIPA, Alaninol, and o-Xylene was performed at T(in)=139-145° C., T(jacket)=170-185° C., and T(head)=95-139° C. without fractioning. (S)-MOIPA was obtained in >99% yield. Alaninol was not completely removed from the sump using these conditions. Subsequently, the sump was treated with 85 g of dist. water to generate a saturated sodium chloride solution and to separate phases.

### Comparison Example

455 mL of o-xylene (2.83 mol) and 234.5 mL of sodium methylate (30 wt % in methanol, 1.26 mol) were added to a 0.75 L reactor flask (with Rushton type impeller and pitched-blade impeller) and brought to 100° C. 100 g of L-alaninol (1.33 mol) were added over 60 min while methanol was distilled of. After addition of L-alaninol was finished, methanol was further distilled of until the boiling point of o-xylene was reached. Meanwhile 36 g of o-xylene were added.

The suspension was brought to 85° C. and 62.4 g of methylchloride gas (1.24 mol) were inserted at 85-70° C. Gas insertion was performed above the suspension at 800 rpm with a velocity of 7 g methylchloride/h.

The composition of the product mixture obtained was analyzed.

More than 99.9% of the MOIPA obtained were (S)-MOIPA.

The yield of (S)-MOIPA was 49% (based on methyl chloride).

The ratio of (S)-MOIPA to the byproduct N-Me-MOIPA was 15:1.

The selectivity of (S)-MOIPA was 90%.

1. A process for preparing an ether of formula I: 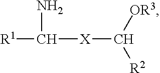
wherein R₁ and R₂ independently from one another are hydrogen or an alkyl group with 1 to 10 C atoms, R3 is an alkyl group with 1 to 10 carbon atoms, and X is a bond or a hydrocarbon group with 1 to 10 carbon atoms,
the process comprising
a) deprotonating an amino alcohol of formula II: 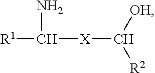
where R₁, R₂ and X have the meaning above, with a metal as deprotonating agent to give an anion of formula III: 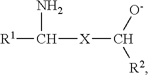
where R₁, R₂ and X have the meaning above; and
b) alkylation of the anion obtained in step a) with an alkylation agent to give the ether of formula I.

2: The process according to claim 1, wherein
R₁ is hydrogen or an alkyl group with 1 to 4 C atoms,
R₂ is hydrogen,
R₃ is an alkyl group with 1 to 4 C atoms, and
X is a bond or an alkylene group with 1 to 10 carbon atoms.

3: The process according to claim 1, wherein
R₁ is a methyl group,
R₂ is hydrogen,
R₃ is a methyl group, and
X is a bond.

4: The process according to claim 1, wherein the compound of formula II is a pure (R) or (S) enantiomer.

5: The process according to claim 1, wherein the metal used as deprotonating agent is an alkali or earth alkali metal.

6: The process according to claim 1, wherein the metal used as deprotonating agent is sodium.

7: The process according to claim 1, wherein step a) is performed at a temperature of at least the melting temperature of the metal used as deprotonating agent.

8: The process according to claim 1, wherein the alkylation agent is selected from alkyl chloride, dialkyl sulfate or dialkyl carbonate.

9: The process according to claim 1, wherein process steps a) and b) are performed in presence of a solvent.

10: The process according to claim 1, wherein process steps a) and b) are performed in presence of a solvent of formula IV
Rₐ—O—(CH₂—O—)ₙR_{b}
wherein
Rₐ and R_{b} are a C1- to C6 alkyl group, and
n is an integral number from 1 to 4.
